(19) United States
(12) Patent Application Publication
(10) Pub. No.: US 2018/0121838 A1
(43) Pub. Date: May 3, 2018

(54) SYSTEMS AND METHODS FOR COORDINATING THE FLOW OF EVENTS IN A HEALTH CARE SETTING USING A WORKFLOW MODULE

(71) Applicant: GetWellNetwork, Inc., Bethesda, MD (US)

(72) Inventors: Bernd Nigmann, Arlington, VA (US); Robin Cavanaugh, Silver Spring, MD (US)

(21) Appl. No.: 15/857,096

(22) Filed: Dec. 28, 2017

Related U.S. Application Data
(63) Continuation of application No. 11/349,194, filed on Feb. 8, 2006, now abandoned.
(60) Provisional application No. 60/650,961, filed on Feb. 9, 2005.

(51) Int. Cl. G06Q 10/06; G06Q 50/22; G06Q 10/10

(52) U.S. Cl. CPC G06Q 10/06; G06Q 50/22; G06Q 10/10; G06Q 10/0633; G06F 19/327

(57) ABSTRACT

A method and system of coordinating events that occur between a time that a person enters a health care setting and a time that the person is discharged from the health care setting by generating and operating a health care setting specific workflow module that includes receiving a selection of one or more predetermined events, and associating one or more workflow processes with the one or more predetermined events. Specifically, one or more workflow processes and associated system component activities includes a plurality of actions that take place after one or more predetermined events occur. The method and system also include implementing the one or more workflow processes using a software program and system component activity after one or more predetermined events occur.

The present application claims priority from U.S. Provisional Patent Application Ser. No. 60/650,961, which was filed on Feb. 9, 2005, and is entitled “Systems and Methods for Coordinating the Flow of Events in a Health Care Facility Using a Workflow Module,” the entirety of which is incorporated herein by reference.

### BACKGROUND OF THE INVENTION

### 1. Field of the Invention

The present invention is related generally to systems and methods for coordinating the flow of events in a health care setting using a workflow module. In particular, the present invention is directed towards systems and methods in which the occurrence of a predetermined event triggers the initiation of software and associated system component activity based workflow processes that include a plurality of predetermined of activities that are to take place after the predetermined event occurs.

### 2. Description of Related Art

In general, when a person enters a health care setting, e.g., a hospital, a doctor's office, or the like, for the purpose of obtaining diagnosis and/or treatment of a medical condition, a series of events will take place between the time that the person enters the health care setting and the time that the person is discharged from the health care setting. Such events may be associated with one or more of a plurality of event categories, such event categories including, but clearly not limited to, admission of the person to the health care setting; selection of a doctor to treat the person; diagnosis of the medical condition associated with the person; selection of the treatment and the diet for the person based on the diagnosis and other relevant factors; monitoring the treatment of the person, e.g., monitoring the person's comfort/discomfort level; and discharge of the person from the health care setting. When an event associated with an event category occurs at the health care setting, employees of the health care setting, e.g., doctors, nurses, orderlies, support staff, and the like, generally need to take action in response to the event.

### SUMMARY OF THE INVENTION

Therefore, a need has arisen for systems and methods for coordinating the flow of events in a health care setting that overcome these and other shortcomings of the related art, as well as accomplishing other goals. An advantage of the present invention is that at least a portion of the events that occur between a time that a person enters a health care setting and a time that the person is discharged from the health care setting may be coordinated, such as automatically, by generating a health care setting specific workflow module.

Specifically, an exemplary embodiment of the present invention is configured to automatically coordinate at least a portion of the events that occur between the time that the person enters the health care setting and the time that the person is discharged from the health care setting, by employing a system and method in which the occurrence of a predetermined event associated with a predetermined event category automatically triggers the initiation of at least one workflow process associated with the predetermined event. The workflow process or processes associated with the predetermined event represent a flow of activities that may, should, or must take place as a result of the occurrence of the predetermined event.

For example, when the person is admitted to the health care setting, generally, the person is registered and a room selected for the person, such room generally including a television or other display and/or communication device. In the present exemplary embodiment, the registration of the person and/or the selection of the room for the person automatically may initiate a first workflow process associated with displaying a safety video to the person, and a second workflow process associated with selecting content, e.g., movies, television, Internet, medical information videos, and/or the like that is available to the person for viewing. In this example, the content that is available to the person for viewing may depend on factors that include, but are not limited to, the age of the person, the gender of the person, the medical condition afflicting the person (once this becomes known), and/or the like. In addition, as the amount of time that the person spends at the health care setting increases, the amount of content available for viewing by the person also may increase.

Moreover, because there are a substantial number of events for which it may be desirable to initiate a workflow process or processes for a particular person, each of the predetermined events may have a particular workflow process or processes associated therewith. For example, in the present invention, the occurrence of a first predetermined event associated with a person may initiate a first workflow process or processes (and associated system component activity), and the occurrence of a second predetermined event associated with the person may initiate a second workflow process or processes (and associated system component activity) that is different than the first workflow process or processes. In addition, depending on when various predetermined events occur, the implementation of multiple workflow processes and system component activity associated with a single person may overlap with each other.

### BRIEF DESCRIPTION OF THE DRAWINGS

For a more complete understanding of the present invention, the needs satisfied thereby, and the objects, features, and advantages thereof, reference now is made to the following description taken in connection with the accompanying drawings.

FIG. 1 depicts a first exemplary workflow process that may be associated with displaying a safety video to a person, according to an embodiment of the present invention.

FIG. 2a depicts a second exemplary workflow process that may be associated with displaying a safety video to a person, according to an embodiment of the present invention.

FIG. 2b depicts exemplary options that a health care setting may select when the health care setting customizes a workflow process associated with displaying a safety video, according to an embodiment of the present invention.

FIG. 3a depicts an exemplary workflow process that may be associated with assessing/monitoring a current pain level of a person, according to an embodiment of the present invention.

FIG. 3b depicts exemplary options that a health care setting may select when the health care setting customizes a workflow process associated with assessing/monitoring a current pain level of a person, according to an embodiment of the present invention.

FIG. 4 depicts an exemplary workflow process that may be associated with post-discharge medication of a person, according to an embodiment of the present invention.

FIG. 5 shows a block diagram of system components in accordance with an exemplary embodiment of the present invention.

FIG. 6 depicts a block diagram of various computer system components that may be used with an exemplary implementation of a system and method according to an embodiment of the present invention.

### DETAILED DESCRIPTION OF PREFERRED EMBODIMENTS

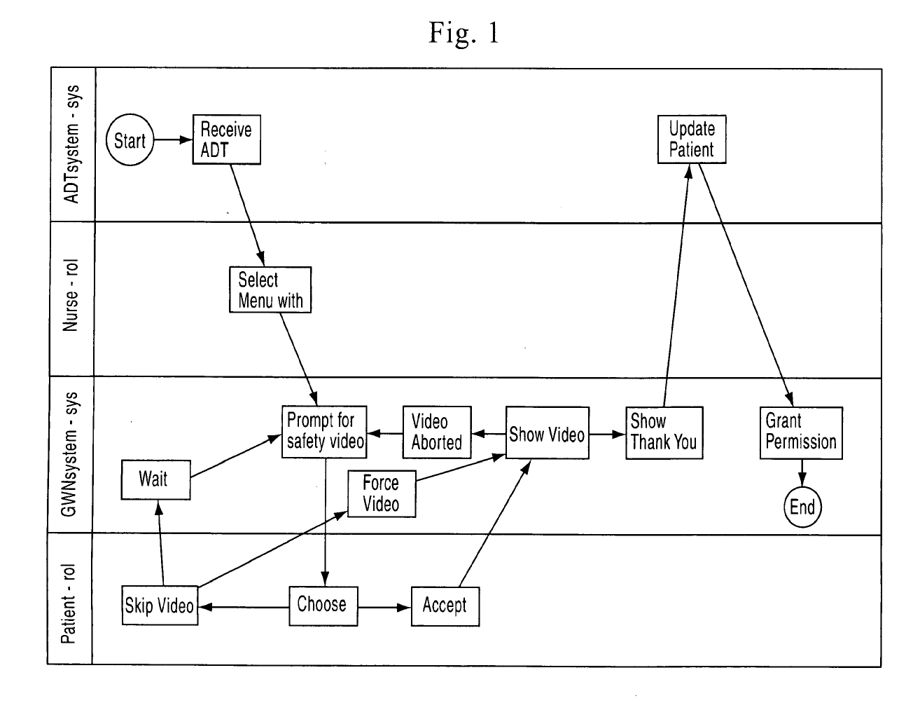

Preferred embodiments of the present invention and their features and advantages may be understood by referring to FIGS. 1-6, like numerals being used for like corresponding parts in the various drawings.

In general, the present invention is configured to allow a provider of the system and method of the present invention to individually customize an overall workflow module and associated system component activity for each health care setting that is a client of the provider. For example, each health care setting can select the extent to which it wishes to coordinate the events that occur between the time that the person enters the health care setting and the time that the person is discharged from the health care setting, e.g., each health care setting can select a fully coordinated system, a partially coordinated system, a minimally coordinated system, or the like. Similarly, each health care setting can select which events qualify as “predetermined events” within their customized system, and also may select the activities included in the workflow process or processes and any system component activity that is/are associated with each predetermined event.

Specifically, the health care setting may select the event categories that it wishes to coordinate, e.g., admission, discharge, and/or monitoring of the person, and also may select the predetermined event or events that are to be included in each event category, such that the health care setting selects which predetermined events they wish to coordinate. For example, the overall workflow module associated with a particular health care setting may include a plurality of workflow packages, and each workflow package may be associated with one of the event categories that the particular health care setting wishes to coordinate, e.g., a first workflow package may be associated with admission of the person, a second workflow package may be associated with monitoring the person, and a third workflow package may be associated with discharge of the person. Moreover, each workflow package may include all of the workflow processes and system component activity associated with predetermined events that are included in the workflow package. As such, the overall workflow module associated with a particular health care setting generally may include all of the workflow processes and system component activity associated with the predetermined events that the particular health care setting wishes to coordinate.

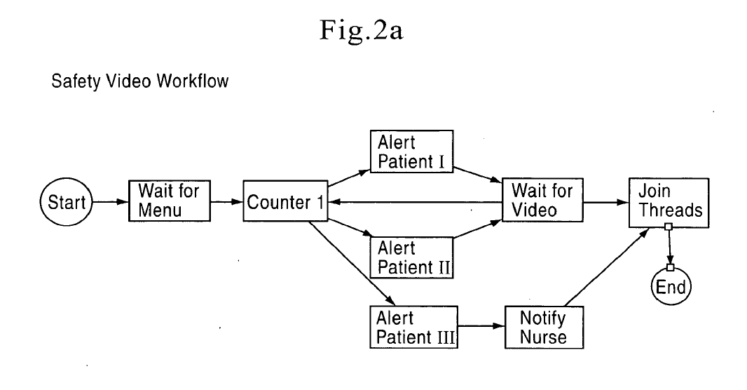

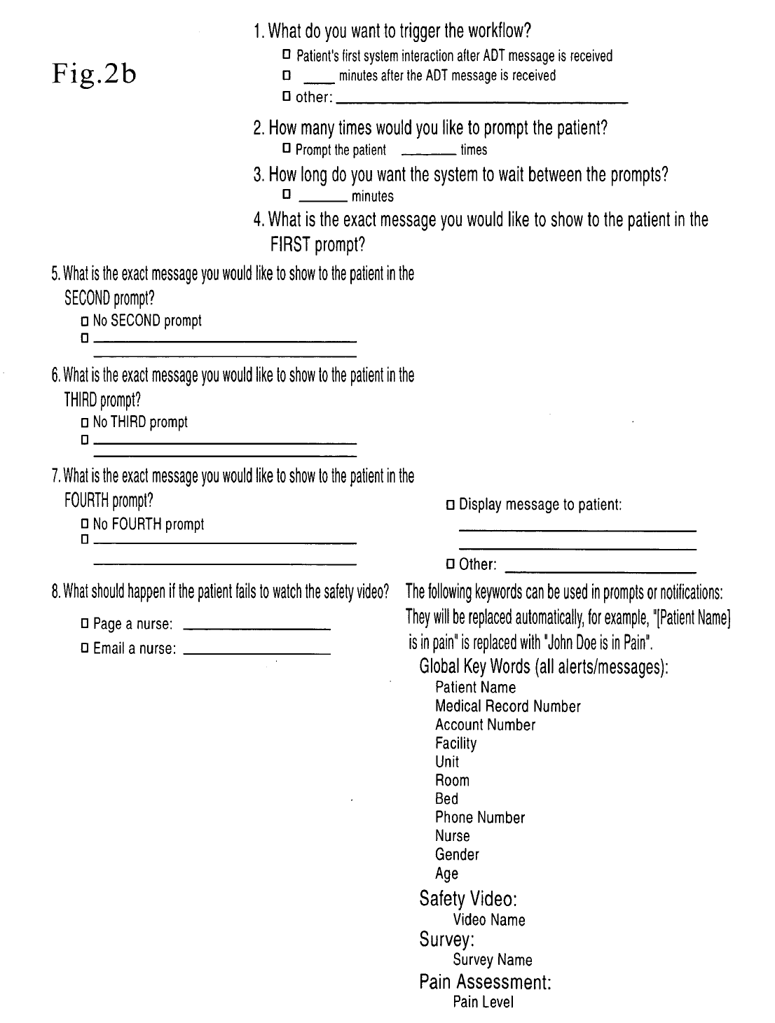

By way of example, FIGS. 1 and 2a depict exemplary workflow processes that may be associated with displaying a safety video to a person, and FIG. 2b depicts exemplary options that the health care setting may select when the health care setting customizes the workflow process associated with displaying the safety video. As noted above, the workflow process associated with displaying the safety video to the person may be included in the workflow package associated with the admission of the person to the health care setting. In this example, the health care setting may register the person at the health care setting and may select a room for the person, which automatically may initiate the workflow process and system component activity associated with displaying the safety video. For example, the workflow process and system components associated with the displaying the safety video may prompt the person to watch the safety video. If the person accepts the option to watch the safety video (e.g., using a system viewing selection component), then the workflow process and system components associated with the safety video may cause the safety video to be displayed to the user, e.g., via a television or other display system component positioned within the room. However, if the person declines the option to watch the safety video (e.g., by using the system viewing selection component), then the option to watch the safety video may be temporarily removed from the television screen.

After the expiration of a predetermined amount of time (e.g., an amount of time that generally is selected by the health care setting when it customizes its workflow module), the option to watch the safety video again may appear on the television screen. If the person accepts the option to watch the safety video, then the workflow process associated with the safety video may display the safety video to the user. However, if the person again declines the option to watch the safety video, then the option to watch the safety video may be temporarily removed from the television screen. This process may continue until the number of times that the person chooses not to watch the safety video is equal to a predetermined number of times (a number of times that generally is selected by the health care setting when it customizes its workflow module), at which point the workflow process and system components associated with the safety video may force the person to watch the safety video, e.g., by refusing, via the system viewing selection component to allow the person to engage in any activities associated with the television until the person watches the safety video.

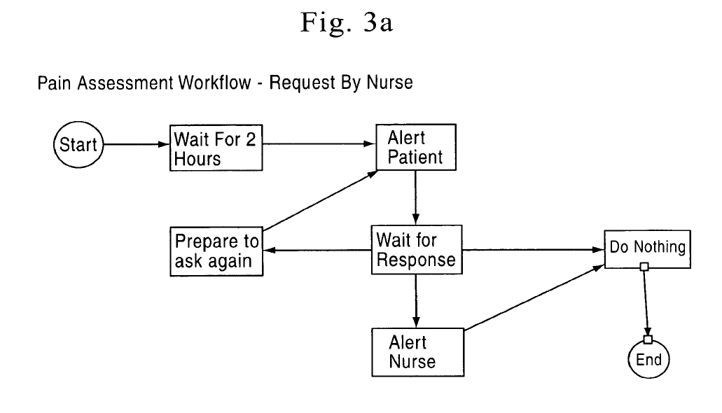

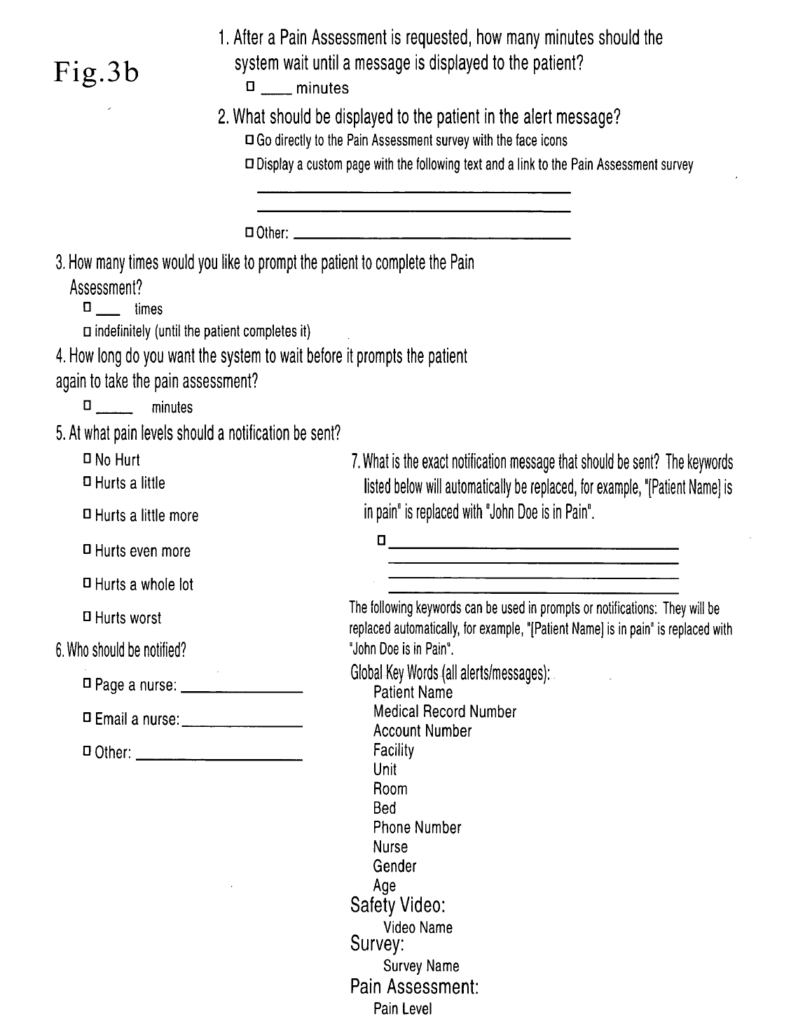

By way of another example, FIG. 3a depicts an exemplary workflow process that may be associated with assessing/monitoring a current pain level of the person, and FIG. 3b depicts exemplary options that the health care setting may select when the health care setting customizes the workflow process associated with assessing/monitoring the current pain level of the person. As noted above, the workflow process, as well as corresponding system components and their activities, associated with assessing/monitoring the current pain level of the person may be included in the workflow package associated with monitoring the person's response to treatment. In this example, a nurse may initiate the workflow process and system component activity associated with assessing/monitoring the current pain level of the person by requesting pain assessment/monitoring of the patient, e.g., after a doctor or nurse provides the person with pain medication, such as morphine. Alternatively, for example, the process and system component status may automatically initiate the workflow process and other system component activity. The workflow process associated with assessing/monitoring the current pain level of the person then may initiate a delay of a predetermined amount of time (an amount of time that generally is selected by the health care setting when it customizes its workflow module) before causing prompts of the person to complete a pain assessment survey, e.g., via the television of other display component and/or other system component and process activity.

By way of example, the pain assessment survey may request that the person select (e.g., via a selection system component) one of the following options with respect to their current pain level, (1) no hurt; (2) hurts a little; (3) hurts a little more; (4) hurts even more; (5) hurts a whole lot; and (6) hurts worse. If the person chooses not to respond to the pain assessment survey, the pain assessment survey temporarily may be removed from the television screen. After the expiration of a predetermined amount of time (e.g., an amount of time that generally is selected by the health care setting when it customizes its workflow module), the workflow process associated with assessing/monitoring the current pain level of the person again may cause as prompting of the person to complete the pain assessment survey. This process may continue until the person completes the pain assessment survey, or until the person chooses not to complete the survey a predetermined number of times, depending on which option the health care setting selects when customizing their workflow module.

After the person completes the pain assessment survey, the system may review the person's responses, and if the person gives a predetermined answer (for example, an answer that generally is selected by the health care setting when it customizes its workflow module, e.g., hurts even more, hurts a whole lot, and/or hurts worst), the system may transmit a notification to a nurse, e.g., via e-mail, page, or the like, so that the nurse can address the person's level of pain, or other system component activity associated with the triggered response could occur (e.g., generation of an alarm or automatic increase in dosage delivered to person, so long as such system component activity is consistent with legal and medical requirements). Whether the system sends such notification to the nurse also may depend on other factors, such as the number of times that the person provides the predetermined answer, e.g., the system may require that the person provide the predetermined answer in consecutive surveys before sending the notification to the nurse. Moreover, after the expiration of a predetermined amount of time (e.g., an amount of time that generally is selected by the health care setting when it customizes its workflow module) from when the person completed the pain assessment survey, the workflow process and system components associated with assessing/monitoring the current pain level of the person again may prompt the person to complete the pain assessment survey.

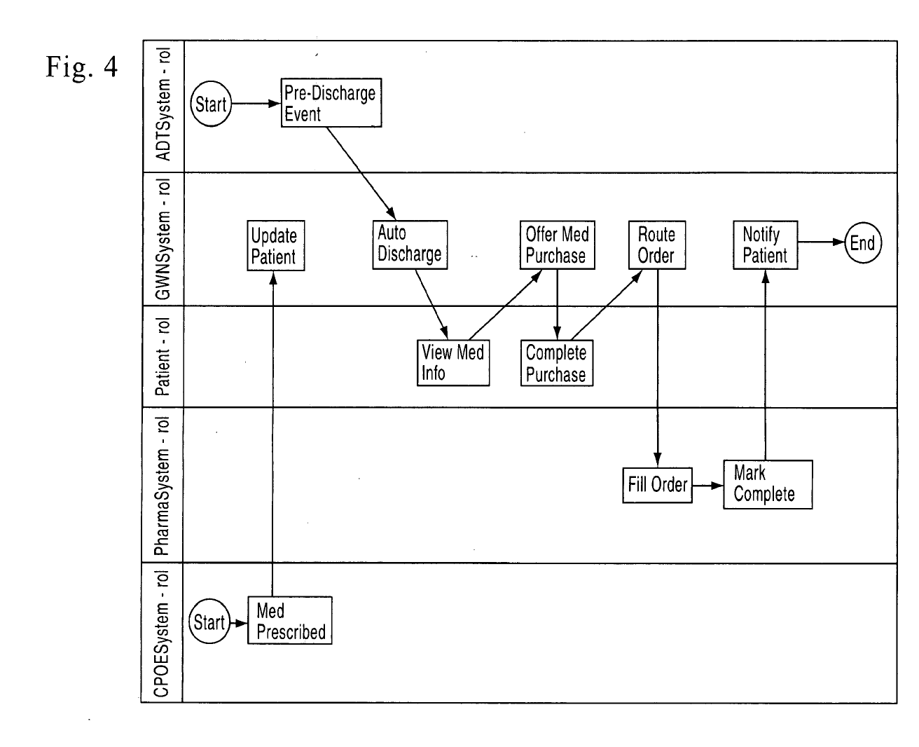

By way of yet another example, FIG. 4 depicts an exemplary workflow process that may be associated with post-discharge medication. The workflow process associated with post-discharge medication may be included in the workflow package associated with discharging the person from the health care setting. In this example, the work-flow process associated with post-discharge medication automatically may be initiated when the doctor prescribes medication that the person should or needs to take after the person is discharged from the health care setting. Specifically, after the doctor prescribes the medication, the workflow process and system components associated with post-discharge medication automatically may prompt the person to purchase the medication. After the purchase is complete, the order automatically may be routed and filled, such that the medication is provided to the person before the person is discharged from the health care setting.

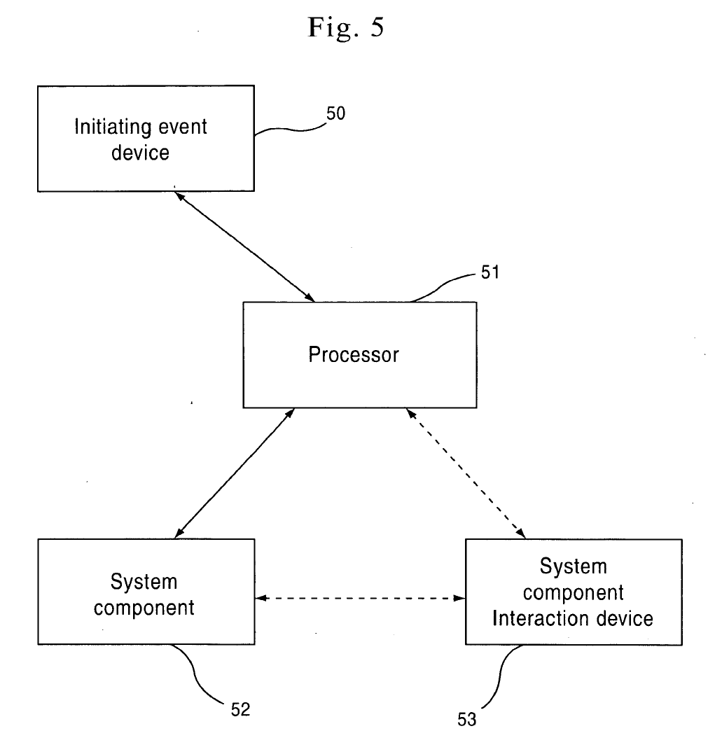

FIG. 5 shows a block diagram of system components in accordance with an exemplary embodiment of the present invention. As shown in FIG. 5, an initiating event input device 50, such as a terminal (e.g., personal computer (PC), minicomputer, microcomputer, mainframe computer, personal digital assistant (PDA) or other handheld device, radiofrequency identification (RFID) device, or other device having input and output features, and, optionally processing capability and display), receives input to begin the process and system component activity. For example, patient admission may constitute an initiating event for which the initiating event input device 50 is used to initiate processes and system activity for that patient.

Communication (e.g., data input and output) with the initiating event input device 50 occurs with a processor 51, such as a central server at the health care setting (e.g., a minicomputer, mainframe computer, PC, a microcomputer, or other device having a processor and repository or capable of accessing a repository, such as a database of patient and workflow process information). Optionally, in one variation, the processor 51 is incorporated into the initiating event input device 50. For example, a user, such as a nurse, may select options relating to a workflow process and system component activity relating to the admitted patient, such as initiation of a safety video procedure and system component operation or initiation of a workflow process and system component activity relating to administering a drug to the patient. The patient admission and selection data received by the initiating event input device 50 is transmitted to the processor 51 and prompts for additional information or other output data from the processor 51 are transmitted to the initiating event input device 50. Alternatively, for example, initiating of the admission data, for example, could occur automatically via the initiating event input device 50, such as by reading RFID information from a patient RFID device. Communication occurs, for example, via wired, wireless, or infrared communication devices and features.

The processor 51 communicates with one or more system components 52, such as a television or other display system component located in the patient's room. For example, a selected workflow process may automatically prompt the user to watch a safety video or to provide drug response information (e.g., current level of pain).

The system component 52 communicates with a system component interaction device 53, such as a video selection device (e.g., a television remote control) or other mechanism to supply workflow process and system component information. For example, the system component interaction device 53 may include one or more video selection features that allow the user to select whether to view the safety video or to input information regarding response to a drug. Alternatively, for example, the system component interaction device 53 could include a monitoring device for the patient could automatically communicate information relating to the patient to the system component 52 or to the processor 51 (e.g., a stress monitor could determine that the patient was under stress and prompt the patient for pain information, or could communicate the stress information to the processor, which in turn communicates the information to a nurse, for example, via the initiating event input device 50).

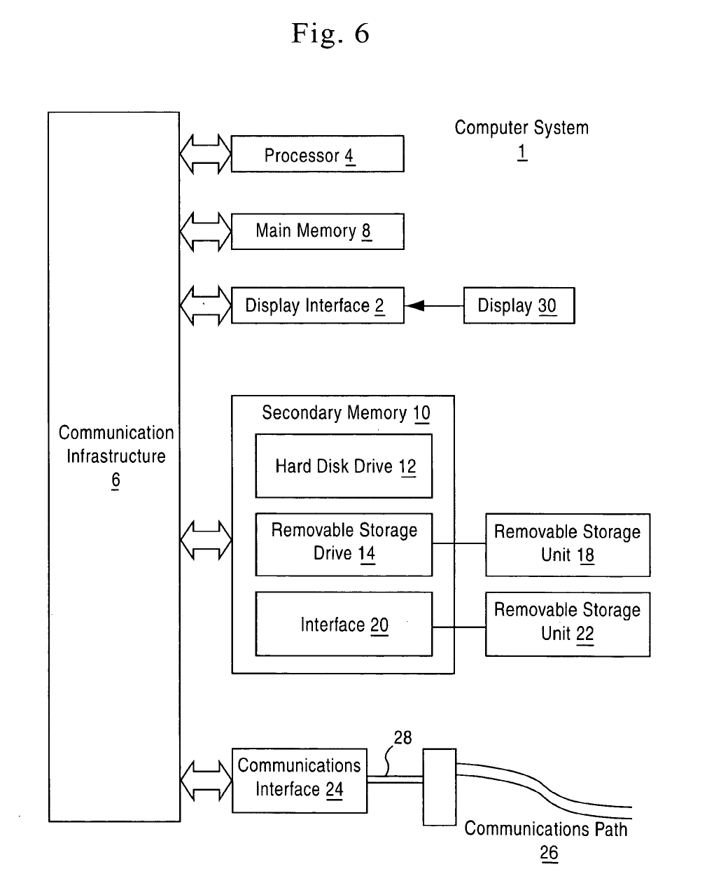

FIG. 6 depicts a block diagram of various computer system components that may be used with an exemplary implementation of a system and method according to an embodiment of the present invention. As shown in FIG. 6, the present invention may be implemented using hardware, software or a combination thereof and may be implemented in one or more computer systems or other processing systems. In one embodiment, the invention is directed toward one or more computer systems capable of carrying out the functionality described above.

Computer system 1 includes one or more processors, such as processor 4. The processor 4 is connected to a communication infrastructure 6 (e.g., a communications bus, cross-over bar, or network). Computer system 1 can include a display interface 2 that forwards graphics, text, and other data from the communication infrastructure 6 (or from a frame buffer not shown) for display on the display unit 30. Computer system 1 also includes a main memory 8, preferably random access memory (RAM), and may also include a secondary memory 10. The secondary memory 10 may include, for example, a hard disk drive 12 and/or a removable storage drive 14, representing a floppy disk drive, a magnetic tape drive, an optical disk drive, etc. The removable storage drive 14 reads from and/or writes to a removable storage unit 18 in a well known manner. Removable storage unit 18, represents a floppy disk, magnetic tape, optical disk, etc., which is read by and written to removable storage drive 14. As will be appreciated, the removable storage unit 18 includes a computer usable storage medium having stored therein computer software and/or data.

In alternative embodiments, secondary memory 10 may include other similar devices for allowing computer programs or other instructions to be loaded into computer system 1. Such devices may include, for example, a removable storage unit 22 and an interface 20. Examples of such may include a program cartridge and cartridge interface (such as that found in video game devices), a removable memory chip (such as an erasable programmable read only memory (EPROM), or programmable read only memory (PROM)) and associated socket, and other removable storage units 22 and interfaces 20, which allow software and data to be transferred from the removable storage unit 22 to computer system 1.

Computer system 1 may also include a communications interface 24. Communications interface 24 allows software and data to be transferred between computer system 1 and external devices. Examples of communications interface 24 may include a modem, a network interface (such as an Ethernet card), a communications port, a Personal Computer Memory Card International Association (PCMCIA) slot and card, etc. Software and data transferred via communications interface 24 are in the form of signals 28, which may be electronic, electromagnetic, optical or other signals capable of being received by communications interface 24. These signals 28 are provided to communications interface 24 via a communications path (e.g., channel) 26. This path 26 carries signals 28 and may be implemented using wire or cable, fiber optics, a telephone line, a cellular link, a radio frequency (RF) link and/or other communications channels. In this document, the terms “computer program medium” and “computer usable medium” are used to refer generally to media such as a removable storage drive 14, a hard disk installed in hard disk drive 12, and signals 28. These computer program products provide software to the computer system 1.

Computer programs (also referred to as computer control logic) are stored in main memory 8 and/or secondary memory 10. Computer programs may also be received via communications interface 24. Such computer programs, when executed, enable the computer system 1 to perform the features of the present invention, as discussed herein. In particular, the computer programs, when executed, enable the processor 4 to perform the features of the present invention. Accordingly, such computer programs represent controllers of the computer system 1.

In an embodiment where the invention is implemented using software, the software may be stored in a computer program product and loaded into computer system 1 using removable storage drive 14, hard drive 12, or communications interface 24. The control logic (software), when executed by the processor 4, causes the processor 4 to perform the functions of the invention as described herein. In another embodiment, the invention is implemented primarily in hardware using, for example, hardware components, such as application specific integrated circuits (ASICs). Implementation of the hardware state machine so as to perform the functions described herein will be apparent to persons skilled in the relevant art(s).

In yet another embodiment, the invention is implemented using a combination of both hardware and software.

While the present invention has been described in connection with preferred embodiments, it will be understood by those skilled in the art that variations and modifications of the preferred embodiments described above may be made without departing from the scope of the invention. Other embodiments will be apparent to those skilled in the art from a consideration of the specification or from a practice of the invention disclosed herein. It is intended that the specification and the described examples are considered exemplary only.

1-21. (canceled)

22. A computer-implemented method of automatically coordinating over a network at least a portion of events that occur between a time that a person enters a health care setting and a time that the person is discharged from the health care setting by generating a health care setting specific workflow module, the computer comprising a processor, a data repository and a user interface and the method comprising:
after the person has entered the health care setting,
receiving, via the user interface of the computer, a selection of at least one predetermined event, the at least one predetermined event being selected from an event category that is selected from the group consisting of: admission of the person to the health care setting, monitoring the person while the person is at the health care setting, and discharge of the person from the health care setting;
associating, via the processor, at least one workflow process with the at least one predetermined event, wherein the at least one workflow process comprises a plurality of predetermined activities that are to take place after the at least one predetermined event occurs, the plurality of predetermined activities being stored in the data repository of the computer;
implementing, via the processor, the at least one workflow process using a software program after the at least one predetermined event occurs;
wherein, if, after an expiration of a predetermined amount of time, at least one of the plurality of predetermined activities does not take place, re-implementing the at least one workflow process; and
wherein the at least one predetermined event is a target event determined in advance.

23. The computer-implemented method of claim 22, wherein selecting the at least one predetermined event comprises:
receiving a selection of at least one event category; and
associating the at least one predetermined event with the at least one event category.

24. A computer-implemented method of automatically coordinating over a network at least a portion of events that occur between a time that a person enters a health care setting and a time that the person is discharged from the health care setting by generating a health care setting specific workflow module, the computer comprising a processor, a data repository and a user interface and the method comprising:
after the person has entered the health care setting,
receiving, via the user interface of the computer, a selection of a first event category, the first event category being selected from the group consisting of: admission of the person to the health care setting, monitoring the person while the person is at the health care setting, and discharge of the person from the health care setting;
receiving a selection of at least one first predetermined event;
associating the at least one first predetermined event with the first event category;
associating at least one first workflow process with the at least one first predetermined event, wherein the at least one first workflow process comprises a first plurality of predetermined activities that are to take place after the at least one first predetermined event occurs, the first plurality of predetermined activities being stored in the data repository of the computer;
implementing the at least one first workflow process using a software program after the at least one first predetermined event occurs;
receiving a selection of a second event category;
receiving a selection of at least one second predetermined event, the second event category being selected from the group consisting of: admission of the person to the health care setting, monitoring the person while the person is at the health care setting, and discharge of the person from the health care setting, the second event category being different from the first event category;
associating the at least one second predetermined event with the second event category;
associating at least one second workflow process with the at least one second predetermined event, wherein the at least one second workflow process comprises a second plurality of predetermined activities that are to take place after the at least one second predetermined event occurs, the second plurality of predetermined activities being stored in the data repository of the computer;
implementing, via the processor, the at least one second workflow process using a software program after the at least one second predetermined event occurs;
wherein, if, after an expiration of a predetermined amount of time, at least one of the first plurality of predetermined activities does not take place, re-implementing the at least one workflow process; and
wherein the at least one first and second predetermined events are target events determined in advance.

25. A non-transitory computer readable memory having recorded thereon statements and instructions for performing a method of automatically coordinating over a network at least a portion of events that occur between a time that a person enters a health care setting and a time that the person is discharged from the health care setting, the statements and instructions for execution by a computer and the method comprising the steps of:
after the person has entered the health care setting,
receiving a selection of at least one predetermined event, the at least one predetermined event being selected from an event category that is selected from the group consisting of: admission of the person to the health care setting, monitoring the person while the person is at the health care setting, and discharge of the person from the health care setting;
associating at least one workflow process with the at least one predetermined event, wherein the at least one workflow process comprises a plurality of predetermined activities that are to take place after the at least one predetermined event occurs;
implementing the at least one workflow process after the at least one predetermined event occurs;
wherein, if, after an expiration of a predetermined amount of time, at least one of the plurality of predetermined activities does not take place, re-implementing the at least one workflow process; and
wherein the at least one predetermined event is a target event determined in advance.

26. The computer readable memory of claim 25, wherein selecting the at least one predetermined event comprises:
receiving a selection of at least one event category; and
associating the at least one predetermined event with the at least one event category.

27. A non-transitory computer readable memory having recorded thereon statements and instructions for performing a method of automatically coordinating over a network at least a portion of events that occur between a time that a person enters a health care setting and a time that the person is discharged from the health care setting, the statements and instructions for execution by a computer and the method comprising the steps of:
after the person has entered the health care setting,
receiving a selection of a first event category, the first event category being selected from the group consisting of: admission of the person to the health care setting, monitoring the person while the person is at the health care setting, and discharge of the person from the health care setting;
receiving a selection of at least one first predetermined event;
associating the at least one first predetermined event with the first event category;
associating at least one first workflow process with the at least one first predetermined event, wherein the at least one first workflow process comprises a first plurality of predetermined activities that are to take place after the at least one first predetermined event occurs, the first plurality of predetermined activities being stored in a data repository of the processing arrangement;
implementing the at least one first workflow process after the at least one first predetermined event occurs;
receiving a selection of a second event category;
receiving a selection of at least one second predetermined event, the second event category being selected from the group consisting of: admission of the person to the health care setting, monitoring the person while the person is at the health care setting, and discharge of the person from the health care setting, the second event category being different from the first event category;
associating the at least one second predetermined event with the second event category;
associating at least one second workflow process with the at least one second predetermined event, wherein the at least one second workflow process comprises a second plurality of predetermined activities that are to take place after the at least one second predetermined event occurs, the second plurality of predetermined activities being stored in the data repository of the processing arrangement;
implementing the at least one second workflow process after the at least one second predetermined event occurs;
wherein, if after an expiration of a predetermined amount of time at least one of the first plurality of predetermined activities does not take place, re-implementing the at least one first workflow process; and
wherein the at least one first and second predetermined events are target events determined in advance.

28. A computer system for automatically coordinating over a network at least a portion of events that occur between a time that a person enters a health care setting and a time that the person is discharged from the health care setting by generating a health care setting specific workflow module, after the person has entered the health care setting, the computer system comprising:
a processor;
a repository;
a user interface:
a module for receiving, via the processor, a selection of at least one predetermined event,
the at least one predetermined event being selected from an event category that is selected from the group consisting of: admission of the person to the health care setting, monitoring the person while the person is at the health care setting, and discharge of the person from the health care setting;
a module for associating, via the processor, at least one workflow process with the at least one predetermined event, wherein the at least one workflow process comprises a plurality of predetermined activities that are to take place after the at least one predetermined event occurs, the plurality of predetermined activities being stored in the repository of the system; and
a module for implementing, via the processor, the at least one workflow process using a software program after the at least one predetermined event occurs;
wherein, if after an expiration of a predetermined amount of time at least one of the plurality of predetermined activities does not take place, re-implementing the at least one workflow process; and
wherein the at least one predetermined event is a target event determined in advance.

29. The computer system of claim 28, wherein selecting the at least one predetermined event comprises:
a module for receiving a selection of at least one event category; and
a module for associating the at least one predetermined event with the at least one event category.

30. The computer system of claim 28, wherein the processor is selected from a group consisting of a personal computer, a minicomputer, a mainframe computer, and a microcomputer.

31. The computer system of claim 28, wherein the processor is coupled to the user interface by a coupling selected from a group consisting of a wired connection, a wireless connection, and a fiberoptic connection.

32. The computer system of claim 28, wherein the user interface comprises a display device.

33. The computer system of claim 28, wherein the user interface includes a video selection device.

34. The computer system of claim 28, wherein the user interface includes a patient monitoring device.
